(19) United States
(12) Patent Application Publication
(10) Pub. No.: US 2012/0157820 A1
(43) Pub. Date: Jun. 21, 2012

(54) METHOD AND SYSTEM FOR DETECTING DISC HAEMORRHAGES

(76) Inventors: Zhou Zhang, Singapore (SG); Jiang Jimmy Liu, Singapore (SG); Wing Kee Damon Wong, Singapore (SG); Joo Hwee Jim, Singapore (SG); Ngan Meng Tan, Singapore (SG); Huiqi Li, Singapore (SG); Shijian Lu, Singapore (SG); Tien Yin Wong, Singapore (SG)

(21) Appl. No.: 13/392,490

(22) PCT Filed: Aug. 24, 2009
(86) PCT No.: PCT/SG2009/000298

§ 371 Date: Feb. 24, 2012

(51) Int. Cl. A61B 6/00

(52) U.S. Cl. 600/407

(57) ABSTRACT

A method for detecting disc haemorrhages in a retinal fundus image. The method includes (a) identifying a ring-shaped region of interest in the retinal fundus image encompassing the optic disc boundary; (b) removing blood vessel regions in the identified region of interest; (c) detecting candidate disc haemorrhages from the removed blood vessels regions in the identified region of interest; and (d) screening the candidate disc haemorrhages. The detected disc haemorrhages may be used to aid in the detection of glaucoma.

### FIELD OF THE INVENTION

The present invention relates to a method and system for detecting disc haemorrhages in a non-stereo retinal fundus image. The method and system can be used to aid the detection of glaucoma.

### BACKGROUND OF THE INVENTION

Glaucoma is a chronic eye condition in which the nerve that connects the eye to the brain (i.e. the optic nerve) is progressively damaged. Patients with early glaucoma do not have visual symptoms whereas patients with a slightly more advanced glaucoma may complain of “tunnel vision” (being able to see only the center) as progression of the disease results in a loss of peripheral vision. Advanced glaucoma at even later stages is associated with total blindness.

There have been two large surveys on glaucoma in Singapore (the Tanjong Pagar Study and the Singapore Malay Eye Study) [1, 2]. These surveys showed that the prevalence of glaucoma among Singaporean adults (40 years and above) is 3-4%, with more than 90% of the patients unaware that they have glaucoma [1, 2].

Worldwide, glaucoma is the second leading cause of blindness. It is projected that 60 million people will contract glaucoma by the year 2010 [3]. Furthermore, glaucoma is responsible for approximately 5.2 million cases of blindness (15% of the total burden of world blindness) [4]. This problem is even more significant in Asia as Asians account for approximately half of the world's glaucoma cases [3]. In addition, because glaucoma is a condition of aging, a larger percentage of people in Singapore and in Asia will be affected due to their aging population.

Early detection of glaucoma is critical to prevent blindness because glaucoma cannot be cured whereas treatment of glaucoma can prevent progression of the disease. However, routine screening for glaucoma in the whole population is not cost effective and is limited by poor sensitivity of current tests. Nevertheless, screening may be useful for high risk individuals, such as first degree relatives of a glaucoma patient, older individuals of age 65 years and above, and elderly Chinese women (who are at risk of contracting angle closure glaucoma).

Currently, there is no systematic way to detect and manage early glaucoma in Singapore. Glaucoma patients are often unaware that they have this condition and hence, often approach ophthalmologists (eye doctors) only when severe visual loss is already present. Unfortunately, treatment at this stage is limited to surgery, is expensive, requires skilled personnel and does not restore vision.

Current methods available for detecting glaucoma include: (1) Assessment of raised intraocular pressure (IOP), (2) Assessment of abnormal visual field and (3) Assessment of damaged optic nerve. The IOP measurement in method (1) is neither specific nor sensitive enough to serve as an effective screening tool whereas visual field testing in method (2) requires special equipment which are only present in tertiary hospitals such as Singapore National Eye Centre, National University Hospitals etc. Although the method of assessing damaged optic nerve (Method (3)) is more promising and superior to the method of measuring IOP (Method (2)) and the method of visual field testing (Method (3)), optic nerve assessment is usually carried out by a trained specialist (ophthalmologist) and such assessment may be subjective. Optic nerve assessment can also be carried out using specialized equipment such as the HRT (Heidelberg Retinal Tomography). However, the availability of such specialized equipment is very limited because of the cost involved. Furthermore, there is usually a shortage of trained operators for such specialized equipment.

Current methods available for the detection of glaucoma also include the following.

The ARGALI (an Automatic cup-to-disc Ratio measurement system for Glaucoma AnaLlysis) system is a system previously developed for glaucoma detection. In ARGALI, the cup-to-disc ratio is used to automatically measure the amount of damage in the optic nerve. The ARGALI system makes use of contour-based methods to determine the cup and disc from a retinal image through an analysis of pixel gradient intensity values throughout the retinal image. Occasionally, where the gradient values are gradual, difficulties in identifying the correct cup can occur.

A Kink-based Analysis method was also previously developed for glaucoma detection. In the Kink-based Analysis method, analysis of blood vessel architecture was used to determine the location of the cup within the optic disc. Using this method, bends in the retinal vasculature over the cup/disc boundary, also known as kinks, were used to determine the physical location of the optic cup. Although this method is non-reliant on color or pallor, there remain challenges in the correct identification of kinks as well as challenges which arise when kinks are absent in some retinal images.

Also previously developed for glaucoma detection is a color intensity based method [8] in which discriminatory color-based analysis was used to determine the location of the cup and disc from a retinal image. A histogram color analysis was performed on the retinal image to determine the threshold cutoff between the cup and the disc. To determine the location of the disc, statistical analysis of the pixel intensities was performed on different features of the retinal image. However, the accuracy of the results obtained from the color intensity based method as compared to clinical ground truth was not assessed.

Methods which make use of information from stereo photographs for the determination of the optic cup and disc have also been developed [9, 10]. While some of the results from these methods seem promising, one disadvantage of these methods is that stereoscopic photography (as opposed to monocular photography used in the ARGALI and Kink-based method) demands specific hardware and requires specialized training. This may render glaucoma detection methods, which use stereoscopic photography, unsuitable for mass screening.

### SUMMARY OF THE INVENTION

The present invention aims to provide a new and useful automatic method and system for detecting glaucoma.

In general terms, the present invention proposes that medically derived landmarks, such as disc haemorrhages, are automatically derived from a monocular image, for use in detecting glaucoma. In some embodiments, this technique is integrated into a method and system for detecting glaucoma using other techniques, so as to improve the accuracy of the glaucoma detection.

While it is true that, in addition to the Cup-to-disc ratio (CDR), it is already known for various grading characteristics to be assessed by clinicians during clinical optic nerve head (ONH) examination, and taken into account in glaucoma detection, and that one such image cue is the presence of disc haemorrhages, in the past such techniques have always employed human involvement, and therefore been not only time-intensive but also subjective. It has not previously been realized that it might be possible to detect disc haemorrhages automatically and with acceptable accuracy.

Specifically, a first aspect of the present invention is a method for detecting disc haemorrhages in a retinal fundus image, the method comprising the steps of: (a) identifying a ring-shaped region of interest in the retinal fundus image encompassing the optic disc boundary; (b) removing blood vessel regions in the identified region of interest; (c) detecting disc haemorrhages from the removed blood vessels regions in the identified region of interest by a colour-based analysis, to identify candidate disc haemorrhages; and (d) screening the candidate disc haemorrhages.

The invention may alternatively be expressed as a computer system for performing such a method. This computer system may be integrated with a device for capturing non-stereo retinal fundus images. The invention may also be expressed as a computer program product, such as one recorded on a tangible computer medium, containing program instructions operable by a computer system to perform the steps of the method.

### BRIEF DESCRIPTION OF THE FIGURES

An embodiment of the invention will now be illustrated for the sake of example only with reference to the following drawings, in which:

FIGS. 1(a)-(b) respectively illustrate the locations of disc haemorrhages in colour and red-free retinal fundus images;

FIG. 2 illustrates a flow diagram of a method 200 according to the invention for performing an automatic detection of disc haemorrhages;

FIG. 3 illustrates images obtained after each sub-step of step 202 of method 200;

FIGS. 4(a)-(c) illustrate images obtained after each sub-step of steps 204 and 206 of method 200;

FIG. 5 illustrates images obtained after performing each sub-step of step 208 of method 200;

FIG. 6(a) illustrates the image obtained from step 210 of method 200 and FIG. 6(b) illustrates the image obtained after step 212 of method 200 is performed on the image of FIG. 6(a);

FIG. 7 illustrates images with disc haemorrhages detected using method 200.

### DETAILED DESCRIPTION OF THE EMBODIMENTS

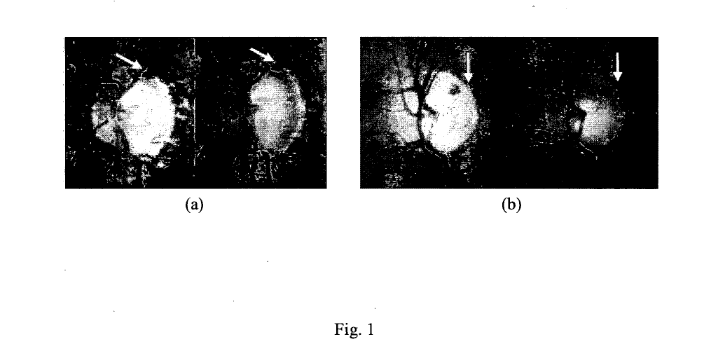

Referring to FIG. 1, the locations of disc haemorrhages in colour and red-free retinal fundus images are indicated by the arrows. Disc haemorrhage is a significant negative prognostic factor in glaucoma [14]. Haemorrhages on or crossing the optic disc have been reported to precede both retinal nerve fiber layer damage and visual field loss in subjects with glaucoma or ocular hypertension. Introducing disc haemorrhage detection into the glaucoma detection system can hence provide a more robust detection of glaucoma. For instance, some glaucomatous retinal nerve heads do present an ordinary CDR and in such cases, landmarks such as the disc haemorrhages will be an important cue for glaucoma detection.

Rarely found in normal eyes, disc haemorrhages are detected in about 4% to 7% in eyes with glaucoma [15], and at least one third of glaucoma patients show a disc haemorrhage at one time or another [13]. Disc haemorrhages are usually dot-shaped when they are within the neural retinal rim and flame-shaped (splinter) when they are on, or adjacent to, the disc margin. Flame-shaped haemorrhages within the Retinal Nerve Fibre Layer (RNFL) that cross the scleral ring in the absence of disc edema (i.e. Drance haemorrhages), are highly suggestive of progressive optic nerve damage [16].

Disc haemorrhages are more common in the early stages of glaucoma. They are usually located in the infero- or supero-temporal disc regions and occur more frequently in normal pressure glaucoma. Depending on their original sizes, they are visible for about 1 to 12 weeks after the initial bleed. A localized RNFL defect and/or NeuroRetinal Rim (NRR) notch may be detected, corresponding to a visual field defect [15].

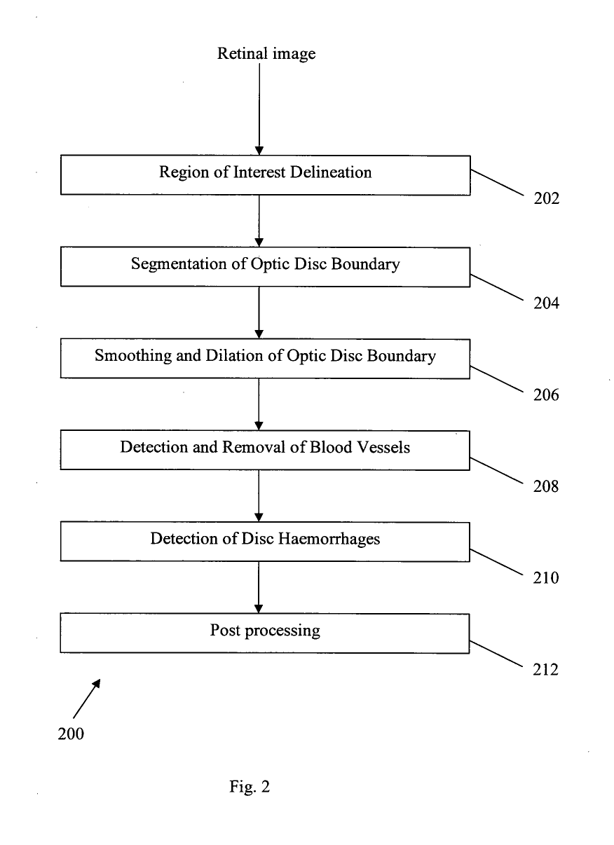

Referring to FIG. 2, the steps are illustrated of a method 200 which is an embodiment of the present invention, and which performs an automatic detection of disc haemorrhages. By the word “automatic”, it is meant that once initiated by a user, the entire process in the present embodiment is run without human intervention. Alternatively, the embodiments may be performed in a semi-automatic manner, that is, with minimal human intervention.

The input to the method 200 is a single non-stereo retinal fundus image. A region of interest is first delineated on the retinal image in step 202. Segmentation of the optic disc boundary is then performed on this region of interest in step 204. Although the segmentation is performed on the region of interest in step 204, it can also be done on the entire image. However, this is not preferred as haemorrhage on other areas of the image are irrelevant to method 200. The segmented optic disc boundary is then smoothed and dilated in step 206 to obtain a “donut ring” region which represents an updated region of interest. Extraction of blood vessels regions within the updated region of interest is then performed in step 208. This is followed by step 210 which performs disc haemorrhage detection within the updated region of interest. Subsequently, post processing is performed on the detected disc haemorrhages in step 212 to remove possible false positive regions wrongly identified as disc haemorrhages.

Steps 202-212 will now be described in more detail.

Step 202: Region of Interest Delineation

In step 202, a region of interest is delineated on the retinal image using a histogram and intensity based method as described below.

High illumination at the retinal boundary is common in retinal fundus images and affects segmentation. It is usually caused by unbalanced exposure or over exposure. To overcome this problem, in step 202, the illumination effect of the retinal fundus image is analyzed by a histogram based study. In the histogram-based study, a prior analysis of a set of 1500 baseline images was performed. The association between the illumination effect caused by unbalanced exposure and the histogram distribution of each of the 1500 images is quantified using scores ranging from −1 to 1. The retinal fundus image is then scored by matching its histogram with the histograms of the baseline images. This score is referred to as an illumination effect score.

An adaptive mask is then generated based on the analysis and is used to filter the retinal image to remove the high illumination at the retinal boundary. A preliminary mask which is a circle centered at the image center with a diameter equal to the height of the image is first generated. The center of the preliminary mask is then adjusted by shifting it away from the portion of the image which has a higher amount of illumination effect. For example, the center of the preliminary mask is shifted to the right of the image if the left side of the image has a higher amount of illumination effect, and is shifted down if the upper rim of the image is highly illuminated. The distance that the center is shifted is based on the illumination effect score obtained in the histogram-based study and the resulting mask with the shifted center is the adaptive mask used to filter the retinal image to remove the noise caused by unbalanced exposure.

After the high illumination at the retinal boundary is removed, the disc center is then estimated using the intensity-based method which extracts the brightest 0.5% of the pixels in the image and subsequently estimates the disc center as the center of gravity of the brightest 0.5% pixels. The region of interest is then created based on the estimated disc center by defining the region of interest as a square surrounding the optic disc with its center being the estimated disc center.

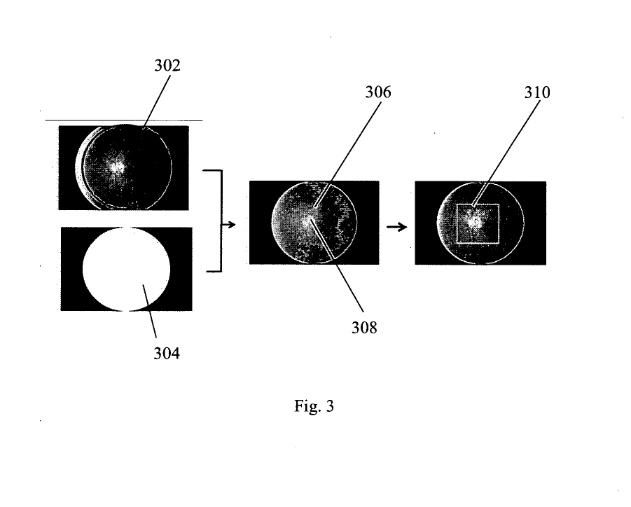

FIG. 3 illustrates images obtained after each sub-step of step 202. As shown in FIG. 3, a circular boundary 302 is obtained after analyzing the illumination effect of the retinal fundus image and an adaptive mask 304 is generated based on the analysis. The image 306 is obtained after the high illumination at the retinal boundary is removed and the disc center 308 is estimated using the intensity-based method. The region of interest (denoted by a square 310) is then created based on the estimated disc center.

Preferably, the region of interest is a square surrounding the optic disc and has a size of 800×800 pixels within an image of 3072×2048 pixels. However, the region of interest may be of a different shape and size.

In step 202 of method 200, the region of interest is delineated using a histogram and intensity based method. However, the delineation of the region of interest may be achieved by other segmentation methods, for example, edge detection methods, region growing methods or model based segmentation methods.

### Steps 204 and 206: Segmentation, Smoothing and Dilation of Optic Disc Boundary

In steps 204 and 206, the optic disc boundary is segmented, smoothed and dilated to obtain an updated region of interest.

In step 204, a variational level-set algorithm [11] is first applied to the region of interest obtained in step 202 to detect the optic disc boundary. This is performed using the optimal colour channel as determined by the colour histogram analysis and edge analysis. The variational level-set algorithm is based on global optimization concepts which analyze the entire region of interest in order to find the globally optimum boundary for the disc. The advantage of using the variational level set algorithm is that it delineates the re-initialization by introducing an energy function consisting of an internal term that keeps the level set function near the signed distance function, as well as an external term that moves the contours towards objects in an image. In step 204, the red channel was utilized as it was observed that better contrast existed between the optic disc and non-disc areas in the red channel as compared to the other channels.

During segmentation, it was observed that the detected contour was often uneven due to the influence of blood vessels across the boundary of the disc, causing inaccuracies in the detected disc boundary, known as leakages. Despite the use of a global optimization technique, the disc boundary detected by the level-set algorithm may not represent the actual shape of the disc, as the disc boundary can be affected by a remarkable number of blood vessels entering the disc. This can often result in sudden changes in curvature. To avoid this, in step 206, ellipse fitting [12] is applied to reshape the disc boundary detected in step 204 so as to smooth it.

Further in step 206, the neuron-retinal rim area is segmented based on the smoothed optic disc boundary and a “donut ring” is generated using disc boundary dilation which dilates the smoothed disc boundary into a “donut ring” with a width set as a fraction of the disc diameter. In step 206, the width of the “donut ring” is set as ⅓ of the disc diameter. The “donut ring” area is the updated region of interest and the disc haemorrhage detection will be subsequently performed in this updated region of interest.

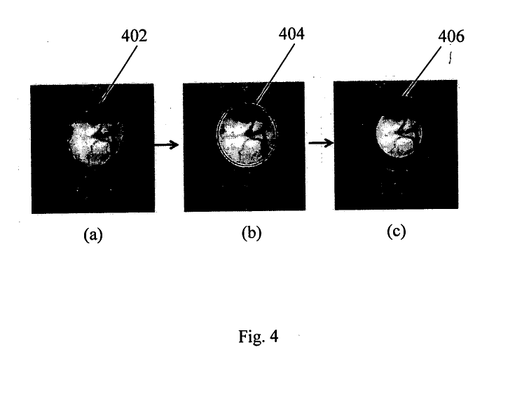

FIGS. 4(a)-(c) illustrate the images obtained after each sub-step of steps 204 and 206. FIG. 4(a) shows the boundary 402 detected using the level set method whereas FIG. 4(b) shows the boundary 404 obtained after boundary smoothing using ellipse fitting. FIG. 4(c) shows the “donut ring” region 406 in which disc haemorrhage detection will be performed subsequently.

In step 204 of method 200, segmentation of the optic disc boundary is performed using the variational level set method. However, other methods such as clustering methods, histogram-based methods, edge detection methods, region growing methods and graph partitioning methods may also be applied to segment the optic disc boundary.

Step 208: Detection and Removal of Blood Vessels

In step 208, a first dilated image is obtained after applying edge detection in the green and grey channels of the retinal image to detect and remove the blood vessels. A grey channel is formed when the RGB retinal image is converted to a grey-scale image. In step 208, edges are detected in the green and grey channels as these edges represent the centerlines of the blood vessels. The green and grey channels are preferred since both green and grey channels are sensitive to the color red. However, it may be possible to use other channels as well. The detected edges are then dilated to form the pixels of the blood vessels and are then removed. Next, a second dilated image comprising an outline of the optic disc region (with finer particles removed by filling up holes which are of a size below a predetermined size) is obtained after applying edge detection in the red channel of the retinal image. The red channel is used for obtaining the second dilated image as the haemorrhage and blood vessel pixels (red pixels) are excluded from the results of the edge detection in the red channel. The results from the individual channels (i.e. the first and second dilated images) are then summed together to remove the blood vessels regions and the summed image is then masked with the updated region of interest obtained in step 206. A resultant image is hence obtained which does not contain the blood vessels regions in the updated “donut-ring” region since the blood vessels in the image have been removed.

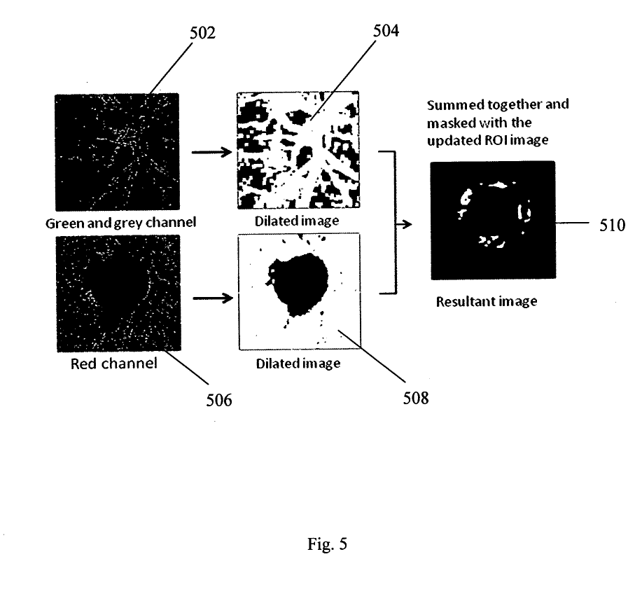

FIG. 5 illustrates the images obtained after performing each sub-step of step 208. A first dilated image 504 is obtained after performing edge detection on the green and grey channels 502 of the retinal image whereas a second dilated image 508 is obtained after performing edge detection on the red channel 506 of the retinal image. The resultant image 510 is obtained after summing up the first and second dilated images 504 and 508, and masking the summed image with the updated region of interest obtained in step 206.

In step 208 of method 200, the detection of blood vessels is performed using an edge detection method. However, in other embodiments, blood vessel detection can be achieved by other means. There are several categories of blood vessel detection algorithms. Model-based approaches include deformable models, parametric models and template matching. Tracking-based approaches require user interaction and hence are preferably not applied in the embodiments of the present invention. Artificial intelligence-based approaches are knowledge-based and require a pre-defined set of rules. Other approaches comprise pattern recognition approaches, including watershed segmentation, skeletonization, multi-scale approaches, centerline extraction and morphological approaches etc.

Step 210: Detection of Disc Haemorrhages

In step 210, disc haemorrhages are detected using a knowledge based approach.

The knowledge based approach employs the knowledge that disc haemorrhages must cross or conjunct with the locations of blood vessels and the knowledge that regions comprising disc haemorrhages are of the highest intensity in the red channel whereas they are of the lowest intensity in the red-free channel.

In step 210, disc haemorrhages are detected from the removed blood vessels regions obtained from step 208 by first computing a histogram for all the pixels of the removed blood vessels regions in the red channel of the retinal fundus image and a histogram for all the pixels of the removed blood vessels regions in the red-free channel of the retinal fundus image. Next, the peaks and valleys of the histograms are used to locate the pixel clusters having the highest intensity in the red channel and the lowest intensity in the red-free channel. These pixel clusters are detected as the disc haemorrhages.

Step 212: Post Processing

Histogram-based intensity extraction in step 210 may pick up more than one location for possible haemorrhage spots (candidate disc haemorrhage areas). Therefore, in step 212, post processing is performed on the disc haemorrhages detected in step 210 to filter possible false positive disc haemorrhages regions. This is performed based on the knowledge that the chances of having more than one disc haemorrhage in a retinal image is very low and the size of a disc haemorrhage is above a predefined value. The predefined value can range from 80 to 275 pixels. This range is based on clinical knowledge.

In step 212, the size of each candidate disc haemorrhage area is checked and candidate disc haemorrhage areas with sizes falling below the predefined value are filtered. Next, the rule that there can only be one disc haemorrhage in each retinal image is applied to retain only the disc haemorrhage with the largest size.

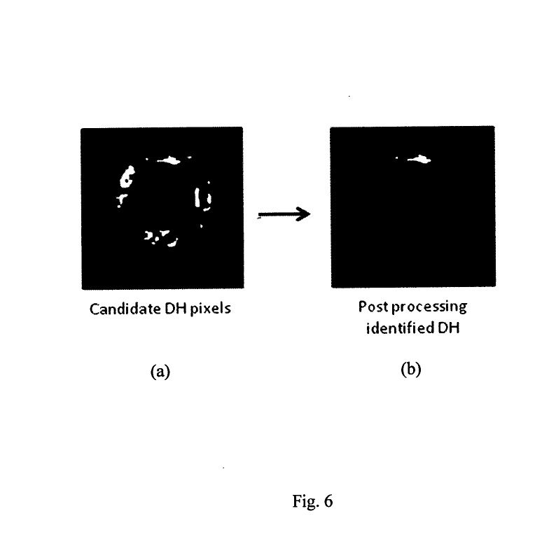

FIG. 6(a) illustrates the image obtained from step 210 containing the candidate disc haemorrhage pixels forming candidate disc haemorrhage areas whereas FIG. 6(b) illustrates the image after post processing is performed on the image of FIG. 6(a).

Experimental Results

A total of 71 images were obtained from the Singapore Malay Eye Study, a survey conducted by the Singapore Eye Research Institute (SERI), for the experiment. This cohort study has enrolled 4.5% of the Singapore population.

The images were analyzed by a senior ophthalmologist from SERI and were assessed for the presence of glaucoma and disc haemorrhages. This assessment by the ophthalmologist was then used as the ground truth in the experiment. According to the ophthalmologist's assessment, disc haemorrhages were found to be present in 11 images whereas they were found to be absent in the remaining 60 images.

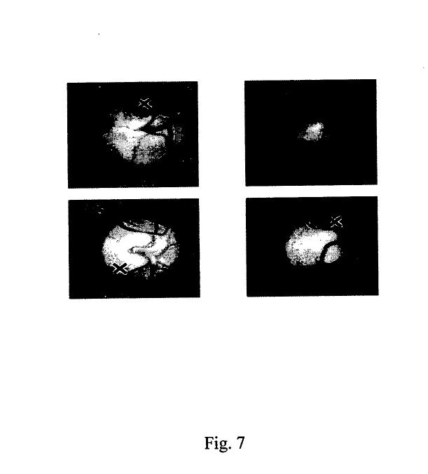

FIG. 7 illustrates four images with disc haemorrhages (represented by the crosses) detected using method 200 whereas Table 1 shows the results obtained using method 200. In Table 1, DH (11) indicates that 11 retinal images contain disc haemorrhage according to the ophthalmologist's assessment whereas Normal (60) indicates that 60 retinal images do not contain disc haemorrhage according to the ophthalmologist's assessment. DH_p and Normal_p respectively indicate the number of retinal images with and without disc haemorrhages as determined by method 200.

As shown in Table 1, 10 out of the 11 images containing disc haemorrhages were correctly identified using method 200 whereas 8 out of the 60 images not containing disc haemorrhages were wrongly identified as containing disc haemorrhages (i.e. false positives). The specificity and sensitivity of method 200 according to this experiment was found to be 86.7% and 90.9% respectively.

TABLE 1

| | DH (11) | Normal (60) |
|---|---|---|
| DH_p | 10 | 8 |
| Normal_p | 1 | 52 |

Automatic detection of disc haemorrhages is challenging due to the interweavement of disc haemorrhages with blood vessels and surrounding tissues around the optic disc. The results of the experiment show that the method 200 is capable of overcoming the difficulties in the automatic detection of disc haemorrhage to achieve a fairly accurate detection of disc haemorrhages.

By applying method 200 to retinal images, the locations of disc haemorrhages in the retinal images can be found and can in turn be used to determine the risk of glaucoma. In one example, the risk of glaucoma is set as high if a disc haemorrhage is located in the retinal image. Alternatively, the locations of the disc haemorrhages in the retinal images can be integrated with other indicators of glaucoma, for example a high cup-to-disc ratio, to improve the accuracy of glaucoma detection. In one example, the risk of glaucoma based on the presence of a disc haemorrhage in the retinal image is combined with the risk of glaucoma based on the cup-to-disc ratio obtained using the ARGALI method to obtain a risk of glaucoma.

Although only the detection of disc haemorrhages is described above, other images cues such as ‘ISNT Rule’ and peripapillary atrophy may also be used to aid in the assessment of glaucoma. Such image cues complement methods such as the ARGALI method which calculates a cup-to-disc ratio since not all instances of glaucoma can be detected via the cup-to-disc ratio. Furthermore, by detecting multiple image cues, the risk of glaucoma can be obtained with a higher confidence.

Embodiments of the present invention hence present an innovative framework for glaucoma analysis and detection from non-stereo retinal fundus images. The use of non-stereo retinal fundus images enables increased functionality on lower-cost equipment.

Computer-aided diagnosis of glaucoma via knowledge-based landmark selection can be achieved using the embodiments of the present invention. Furthermore, by making use of grading characteristics commonly referred by medical domain experts in landmark selection, clinical expertise can be embedded into the system for detecting glaucoma.

In addition, in method 200, a region of interest is first delineated on the retinal image before further processing in subsequent steps is performed. This helps to reduce the computational cost as well as improve segmentation accuracy.

A further advantage of the method 200 is that it can be readily incorporated into currently available instruments for ocular screening, such as glaucoma screening, without extensive modifications.

Comparison with Prior Arts

A comparison between the embodiments of the present invention described above, and prior arts [6-10] is summarized in Table 2.

TABLE 2

| | Technology | | | |
|---|---|---|---|---|
| | of cup | Imaging | Feature | |
| | detection | Technology | inclusion | Limitation |
| Prior | Contour | Monocular | Gradient | Indistinct and |
| Art [6] | based on | | features | gradual pallor |
| | gradient | | | gradients |
| | analysis | | | |
| Prior | Automated | Monocular | Retinal | Kinks may not |
| Art [7] | kink | | vasculature | be present in all |
| | identification | | features | retinal images |
| | system | | | |
| Prior | Discrimi- | Monocular | Pixel color | Color |
| Art [8] | natory | | | information may |
| | analysis- | | | be inaccurate |
| | based | | | |
| | Thresholding | | | |
| Prior | Modified | Stereoscopic | Pixel | Requires pre- |
| Art [9] | deformable | | features | processing of |
| | model | | | stereo retinal |
| | technique | | | images to obtain |
| | | | | cup information |
| Prior | Pixel feature | Stereoscopic | Pixel | Relies on |
| Art [10] | classification | | features | features from |
| | | | | stereo color |
| | | | | retinal images |
| Embodi- | Knowledge- | Monocular | Multiple | Although the |
| ments of | based | | clinically | embodiment is |
| the | landmark | | established | successful in |
| present | detection + | | landmarks | identifying disc |
| invention | image | | | haemorrhages, |
| | analysis | | | enhancing it to |
| | | | | incorporate |
| | | | | other landmarks |
| | | | | depends on |
| | | | | clinical expertise |
| | | | | to identify some |
| | | | | landmarks |

### REFERENCES

1. S. Y. Shen et al., “The prevalence and types of glaucoma in malay people: the Singapore Malay eye study,” Invest Ophthalmol Vis Sci, 2008. 49(9): p. 3846-51.
2. P. J. Foster et al., “The prevalence of glaucoma in Chinese residents of Singapore: a cross-sectional population survey of the Tanjong Pagar district,” Arch Ophthalmol, 2000. 118(8): p. 1105-11.
3. H. A. Quigley, A. T. Broman, “The number of people with glaucoma worldwide in 2010 and 2020,” Br J Ophthalmol, 2006. 90(3): p. 262-7.
4. B. Thylefors and A. D. Negrel, “The global impact of glaucoma,” Bull World Health Organ, 1994. 72(3): p. 323-6.
5. D. H. Sim and L. G. Goh, “Screening for glaucoma in the Chinese elderly population in Singapore,” Singapore Med J, 1999. 40(10): p. 644-7.
6. J. Liu, D. W. K. Wong, J. H. Lim, H. Li, N. M. Tan, Z. Zhang, T. Y Wong, R. Lavanya, “ARGALI: An Automatic Cup-To-Disc Ratio Measurement System For Glaucoma Analysis Using Level-Set Image Processing”, 13th International Conference on Biomedical Engineering (ICBME2008), December 2008.
7. D. W. K. Wong, J. Liu, J. H. Lim., H. Li, X. Jia, F. Yin, T. Y. Wong,“Automated detection of kinks from blood vessels for optic cup segmentation in retinal images”, accepted for SPIE Medical Imaging 2009, February 2009.
8. N. Inoue, K. Yanashima, K. Magatani, and T. A. K. T. Kurihara, “Development of a simple diagnostic method for the glaucoma using ocular Fundus pictures,” 27th Annual International Conference of the, Engineering in Medicine and Biology Society, 2005.
9. M. D. Abramoff, W. L. M. Alward, E. C. Greenlee, L. Shuba, C. Y. Kim, J. H. Fingert, and Y. H. Kwon, “Automated Segmentation of the Optic Disc from Stereo Color Photographs Using Physiologically Plausible Features,” Investigative Ophthalmology and Visual Science, vol. 48, pp. 1665, 2007.
10. J. Xu, O. Chutatape, E. Sung, C. Zheng, and P. Chew Tec Kuan, “Optic disc feature extraction via modified deformable model technique for glaucoma analysis,” Pattern Recognition, vol. 40, pp. 2063-2076, 2007.
11. C. Li, C. Xu, C. Gui, M. D. Fox, “Level set evolution without re-initialization: a new variational formulation,” in Proceedings of the 2005 IEEE Computer Society Conference on Computer Vision and Pattern Recognition, pp. 430-436 vol. 1, June 2005.
12. A. Fitzgibbon, M. Pilu, and R. B. Fisher, “Direct least square fitting of ellipses,” IEEE T Pattern Anal, vol. 21, pp. 476-480, May 1999.
13. Gloster J. “Incidence of optic disc haemorrhages in chronic simple glaucoma and ocular hypertension”, Br J Ophthalmol. 1981 July; 65(7): 452-456
14. S. Kim, K. Park. The Relationship between Recurrent Optic Disc Hemorrhage and Glaucoma Progression Ophthalmology, Volume 113, Issue 4, Pages 598-602
15. Jost B. Jonas, Wido M. Budde; Glaucoma diagnosis—optic disc ssessment; http://www.glaucomaworld.net N°18-JANUARY 2000.
16. DOUGLAS J. RHEE, M. D.; Optic Neuropathy with Pathological Cupping; Massachusetts Eye and Ear Infirmary 2006 VOLUME 1, ISSUE 1.

1. A method for detecting disc haemorrhages in a retinal fundus image, the method comprising the steps of:
(a) identifying a ring-shaped region of interest in the retinal fundus image encompassing the optic disc boundary;
(b) removing blood vessel regions in the identified region of interest;
(c) detecting disc haemorrhages from the removed blood vessels regions in the identified region of interest by a colour-based analysis, to identify candidate disc haemorrhages; and
(d) screening the candidate disc haemorrhages.

2. A method according to claim 1, wherein step (a) comprises the sub-steps of:
(i) identifying an initial region of interest;
(ii) estimating the position of the optic disc boundary in the initial region of interest; and
(iii) dilating the estimated optic disc boundary to obtain the ring-shaped region of interest.

3. A method according to claim 2, wherein step (i) comprises the sub-steps of:
estimating a disc center of the retinal fundus image; and
creating the initial region of interest based on the estimated disc center.

4. A method according to claim 3, wherein step (i) further comprises the sub-step of filtering the retinal fundus image to remove high illumination at a retinal boundary of the retinal fundus image prior to estimating the disc center of the retinal fundus image.

5. A method according to claim 4, wherein the sub-step of filtering the retinal fundus image further comprises the sub-steps of:
analyzing the retinal fundus image by a histogram-based study comprising the sub-steps of:
calculating histograms of a plurality of baseline images and the retinal fundus image;
assigning a score to each of the plurality of baseline images, the score indicating an amount of illumination effect in the baseline image;
comparing the histogram of the retinal fundus image with the histogram of each of the plurality of baseline images; and
assigning a score to the retinal fundus image based on the comparison and the score assigned to each of the plurality of baseline images;
generating an adaptive mask based on the analysis; and
applying the adaptive mask on the retinal fundus image to filter the retinal fundus image.

6. A method according to claim 5, wherein the sub-step of generating an adaptive mask based on the analysis further comprises the sub-steps of:
generating a preliminary mask, the preliminary mask being a circle centered at a center of the retinal fundus image and with a diameter equal to a height of the image;
adjusting the preliminary mask by shifting the center of the preliminary mask away from a portion of the image with a higher amount of illumination effect, the shift being performed by a distance based on the score assigned to the retinal fundus image; and
setting the adaptive mask as the adjusted preliminary mask.

7. A method according to claim 2, wherein the estimated optic disc boundary is smoothed prior to step (iii).

8. A method according to claim 2, wherein step (ii) is performed using a variational level set algorithm.

9. A method according to claim 2 wherein step (ii) is performed only on the red channel of the retinal fundus image.

10. A method according to claim 2, wherein the shape of the initial region of interest identified in step (i) is a square.

11. A method according to claim 1, wherein step (b) further comprises the sub-steps of:
forming a first dilated image by applying edge detection on green and grey channels of the retinal fundus image to detect and remove blood vessels;
forming a second dilated image by applying edge detection on a red channel of the retinal fundus image to obtain an outline of an optic disc region in the retinal fundus image;
summing the first and second dilated images to obtain a summed image; and
masking the summed image with the identified region of interest to remove blood vessels regions in the identified region of interest;

12. A method according to claim 1, wherein the removed blood vessels regions comprise a plurality of pixels and step (c) comprises the sub-steps of:
(ix) calculating a first histogram for the plurality of pixels in the removed blood vessels regions in the red channel of the retinal fundus image;
(x) calculating a second histogram for the plurality of pixels in the removed blood vessels regions in the red-free channel of the retinal fundus image;
(xi) using peaks and valleys of the first and second histograms to locate pixel clusters having the highest intensity in the red channel of the retinal fundus image and the lowest intensity in the red-free channel of the retinal fundus image; and
(xii) detecting the disc haemorrhages as the located pixel clusters.

13. A method according to claim 1, wherein step (d) comprises the sub-steps of:
comparing the size of each candidate disc haemorrhage with a predefined value; and
removing the candidate disc haemorrhage if the size of the candidate disc haemorrhage falls below the predefined value.

14. A method according to claim 13, further comprising the sub-step of:
removing the candidate disc haemorrhage if the size of the candidate disc haemorrhage is not the largest.

15. A method according to claim 1, further comprising:
determining that a high risk of glaucoma exists if at least one disc haemorrhage is detected in the retinal fundus image.

16. A computer system having a processor arranged to perform a method comprising:
(a) identifying a ring-shaped region of interest in the retinal fundus image encompassing the optic disc boundary;
(b) removing blood vessel regions in the identified region of interest;
(c) detecting disc haemorrhages from the removed blood vessels regions in the identified region of interest by a colour-based analysis, to identify candidate disc haemorrhages; and
(d) screening the candidate disc haemorrhages.

17. A computer program product, readable by a computer and containing instructions operable by a processor of a computer system to cause the processor to perform a method comprising:
(a) identifying a ring-shaped region of interest in the retinal fundus image encompassing the optic disc boundary;
(b) removing blood vessel regions in the identified region of interest;
(c) detecting disc haemorrhages from the removed blood vessels regions in the identified region of interest by a colour-based analysis, to identify candidate disc haemorrhages; and
(d) screening the candidate disc haemorrhages.
